(19) United States
(12) Patent Application Publication
(10) Pub. No.: US 2002/0042930 A1
(43) Pub. Date: Apr. 11, 2002

(54) Regulation and manipulation of sucrose content in sugarcane

(76) Inventors: Frederik Coenraad Botha, Stellenbosch (ZA); Jan Hendrik Groenewald, Somerset West (ZA)

Correspondence Address: Piper Marbury Rudnick & Wolfe, P.O. Box 64807, Chicago, IL, 60664-0807, US

(21) Appl. No.: 09/779,237

(22) Filed: Feb. 8, 2001

(30) Foreign Application Priority Data
Feb. 8, 2000 (ZA) 2000/0574

(51) Int. Cl. C12N 15/82; C12N 15/29; C12P 19/04

(52) U.S. Cl. 800/284; 800/286; 536/23.6

(57) ABSTRACT

This invention relates to the regulation and manipulation of sucrose content in a sugar-storing plant, such as sugarcane, by regulating the activity of the PFP enzyme in the plant. It has been found that the down regulation of the PFP enzyme by decreasing the concentration of one of subunits, namely the β-subunit, of the enzyme increases the sucrose content of the plant. In a preferred embodiment of the invention, the activity of the PFP enzyme is down regulated by the introduction of an untranslatable form, or an antisense form of an isolated nucleotide sequence of the invention.

### BACKGROUND OF THE INVENTION

This invention relates to the regulation and manipulation of sucrose content in sugarcane.

The amount of sucrose that accumulates in the culm of sugarcane is a function of sink strength and carbon partitioning between competing metabolic pathways (Whittaker and Botha, 1997). One of these pathways is respiratory flux.

Pyrophosphate-dependent phosphofructokinase (PFP), with the international identification code EC 2.7.1.90, catalyses the reversible conversion of fructose 6-phosphate (F6P) and pyrophosphate (PPi) to fructose 1 ,6-bisphosphate (Fru-1,6-P₂) and inorganic phosphate (Pi) (Carnal and Black, 1979). The levels of both PPi and F6P could potentially control the rate of glycolysis, i.e. respiratory flux (Plaxton, 1996 and references therein). Furthermore, PFP is associated with sink tissues, and could play a role in determining sink strength (Edwards and ap Rees, 1986; Botha et al., 1992; Black et al., 1995). Similar to most other enzymes involved in plant metabolism, PFP activity in sugarcane appears to be controlled, in part, by the expression of the PFP protein (Whittaker and Botha, 1999). However, this is not proof that PFP activity in a plant does alter sucrose content in the plant. Further, previous studies have shown that reduced PFP in transgenic potato and tobacco plants does not lead to different phenotypic characteristics from control plants (Hajirezaei et al 1994; Sonnewald et al 1995).

It is an object of the invention to regulate and manipulate sucrose content in a sugar-storing plant, such as sugarcane.

### SUMMARY OF THE INVENTION

It has been found that sucrose content in a sugar storing plant can be regulated and manipulated by regulating the activity of the PFP enzyme in the plant.

Down regulation of the PFP enzyme by decreasing the concentration of one of the subunits of the enzyme has been found to increase the sucrose content of the plant.

In a preferred embodiment of the invention, the activity of the PFP enzyme is down regulated by the introduction of an untranslatable form or an antisense form of the nucleotide sequence as set out in FIG. 1, a nucleotide sequence which is complementary to the nucleotide sequence of FIG. 1, a variant of the nucleotide sequence of FIG. 1, a portion of the nucleotide sequence of FIG. 1, or a nucleotide sequence which hybridizes to the nucleotide sequence of FIG. 1 under stringent hybridization conditions.

The untranslatable or antisense nucleotide sequence may be introduced to the plant using plant expression vectors such as pUSPc 510 or pASPc 510.

According to the invention an isolated nucleotide sequence comprises:

(i) a nucleotide sequence as set out in FIG. 1;

(ii) a nucleotide sequence which is complementary to the nucleotide sequence of (i);

(iii) a variant of the nucleotide sequence of (i);

(iv) a portion of the nucleotide sequence of (i); or

(v) a nucleotide sequence which hybridizes to the nucleotide sequence of (i) under stringent hybridization conditions.

The nucleotide sequence may be the nucleotide sequence as set out in FIG. 2.

The nucleotide sequence may be in an antisense orientation.

According to another aspect of the invention a gene construct comprises a promoter and nucleotide sequence as defined herein in a sense orientation, the gene construct lacking a translation initiation codon upstream of the nucleotide sequence or possessing an in-frame termination codon directly downstream of the initiation codon.

The gene construct may comprise two promoters.

The promoters may be selected from the CaMV35S and the maize polyubiquitin (UBI) promoters.

According to another aspect of the invention a gene construct comprises a promoter and a nucleotide sequence as defined herein in an antisense orientation.

The gene constructs may be plant expression vectors, pUSPc 510 and pASPc 510 respectively.

According to another aspect of the invention a transformed plant cell comprises a gene construct of the invention.

According to another aspect of the invention a transgenic plant or plant part containing or derived from the transformed plant cell is provided.

The transgenic plant part may be a callus.

The transformed plant cell or transgenic plant or plant part may be characterized by a lower level of the PFP β protein.

The transformed sugarcane cell or transgenic plant or plant part may be characterized by a lower level of PFP activity.

The transformed plant cell or transgenic plant or plant part may be characterized by a higher level of sucrose.

According to another aspect of the invention a method of regulating or manipulating the level of active PFP in a plant cell includes the step of transforming the plant cell with at least one gene construct of the invention.

According to another aspect of the invention a method of maintaining or increasing the sucrose level in sugarcane tissue includes the step of transforming cells of the plant tissue with at least one gene construct of the invention.

According to another aspect of the invention a method of manipulating sucrose metabolism in a plant cell of a sugar-storing plant includes the step of co-transforming the cell with each of the gene constructs of the invention.

The method may involve the alteration of sucrose metabolism in a sugar-storing plant or sugar-storing plant part containing stored sugar.

The plant may be sugarcane.

### BRIEF DESCRIPTION OF THE DRAWINGS

The invention will now be described in more detail, by way of example only, with reference to the accompanying drawings in which:

FIG. 1 is the nucleotide sequence of the 1170 base pair (bp) cDNA fragment of clone PFP#5, containing the 3′ end of the sugarcane PFP-β gene used in the construction of the plant expression vectors pUSPc 510 and pASPc 510;

FIG. 2 is the complete cDNA nucleotide sequence of the sugarcane PFP-β gene;

FIG. 3 is a schematic representation of the plant expression vector, pUSPc 510, containing the 1170 bp PFP-β cDNA fragment in an untranslatable form;

FIG. 4 is a schematic representation of the plant expression vector pASPc 510 containing the 1170 bp PFP-β cDNA fragment in an antisense form;

FIG. 5 is an example of a PCR analysis by which transgenic clones were identified;

FIG. 6 is an example of a northern blot analysis which shows that the 1.2 kb transgene is transcribed in transgenic sugarcane and that the levels of the 2.3 kb endogenous transcript were modulated in the transgenic clones; and

FIG. 7 is an example of a protein blot analysis which shows the changes in the levels of the PFP-β polypeptide in transgenic sugarcane.

### DETAILED DESCRIPTION OF THE INVENTION

It has been found that the sucrose content of a sugar-storing plant can be altered by regulating the activity of the PFP enzyme in the plant. Although this invention describes the regulation of the PFP enzyme in sugarcane, the regulation of the activity of the PFP enzyme in other sugar-storing plants such as sweet sorghum and sugar beet will also affect the sucrose content of the plant.

The PFP enzyme is a heterotetramer with two α and two β-subunits. The β-subunit is the catalytic subunit of the enzyme, and it has been found that removal of the β-subunit increases the sugar content of a sugar-storing plant, namely sugarcane. A similar effect can be attained by altering the α-subunits of the enzyme which are believed to be involved in the regulation of enzyme activity through fructose 2,6-biphosphate (Fru-2,6-P₂).

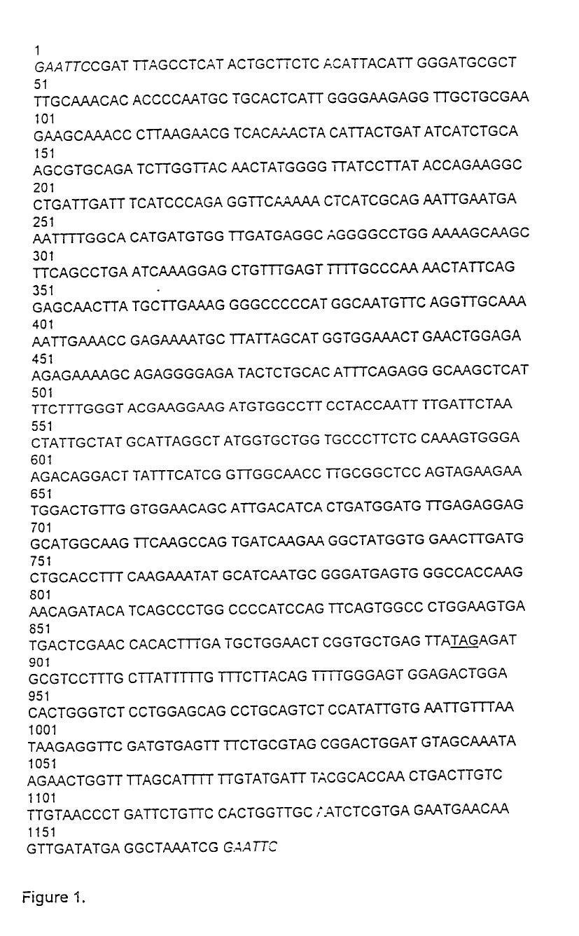

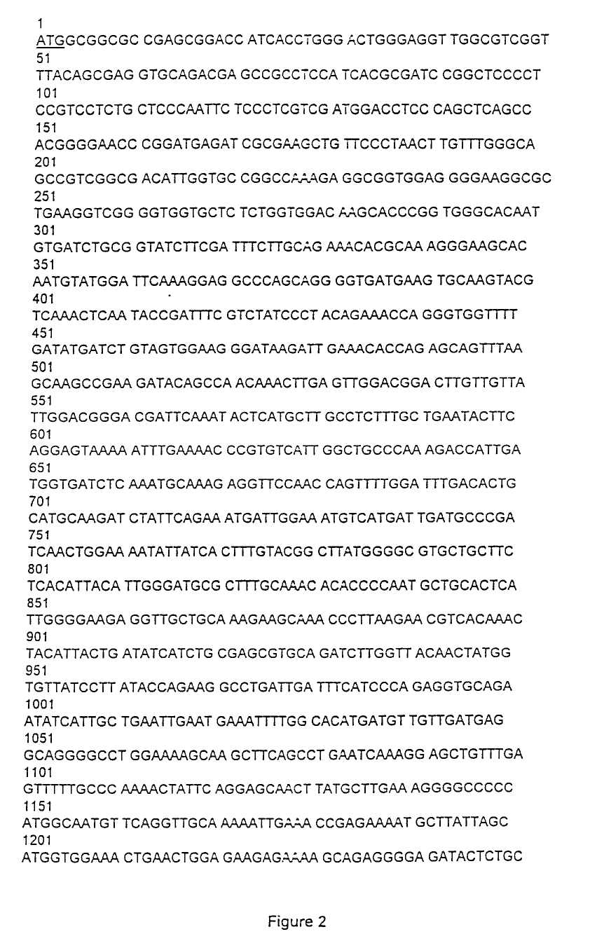

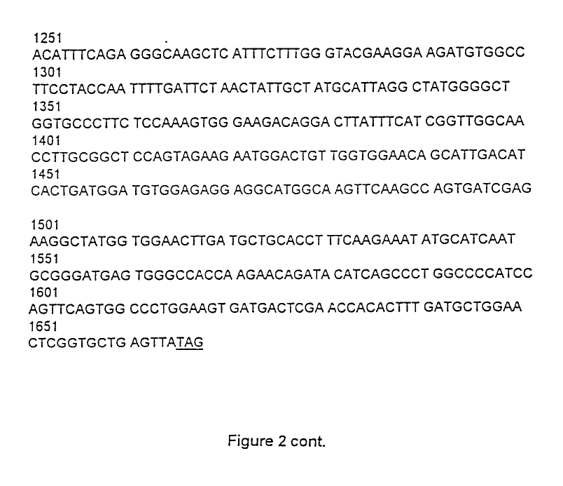

A first step of the invention was the cloning and characterization of a sugar cane PFP-β cDNA fragment. A set of degenerate primers was designed, based on the consensus of the castor bean and potato PFP-β gene sequences. These primers were used to amplify a fragment from sugarcane leafroll RNA which was then used as a probe to screen a sugarcane leafroll cDNA library for putative PFP-β clones. The sequence of the insert of one such clone is shown as an example in FIG. 1. This sequence contains a 1170 bp cDNA fragment. The complete sugarcane PFP-β coding sequence, as shown in FIG. 2, was obtained by sequencing other cDNA and gDNA (genomic DNA) library clones.

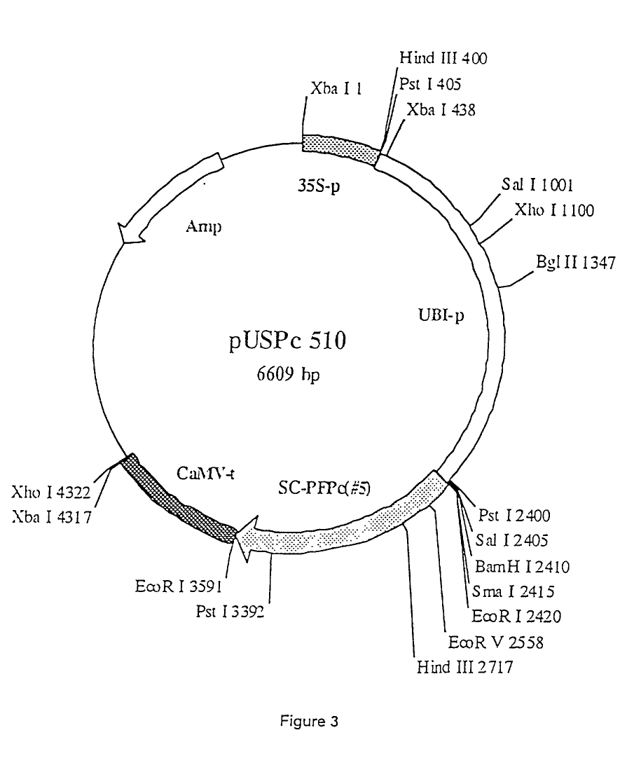

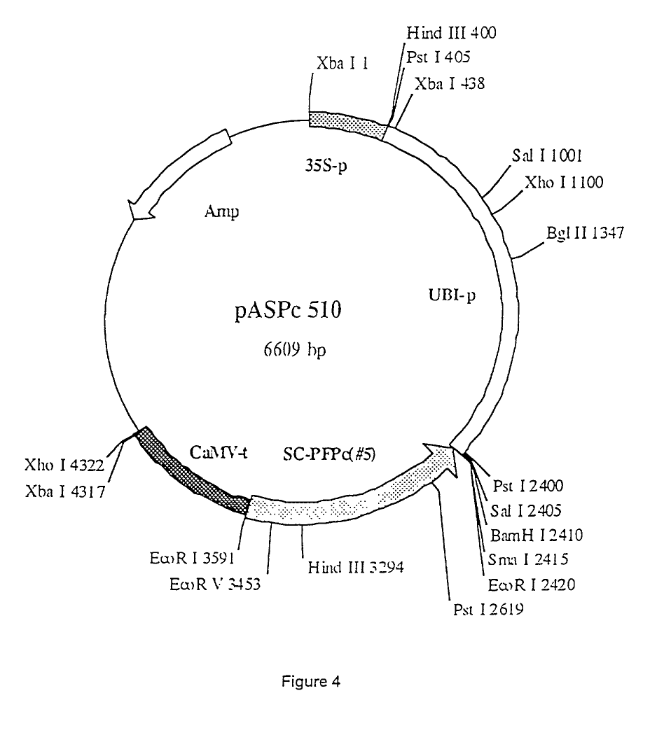

The PFP-β cDNA fragment shown in FIG. 1 was excised and cloned into the plant expression vector pUBI 510 which confers high-level constitutive gene expression in sugarcane cells. One of the vectors, termed pUSPc 510, shown in FIG. 3, contains a fragment in the sense orientation but it lacks a translation initiation codon, and is thus untranslatable. The other vector, termed pASPc 510, shown in FIG. 4, contains a fragment in the antisense orientation.

The vectors pUSPc 510 and pASPc 510 were used to transform cells in a sugarcane callus. Transformed calli were selected and putative transgenic plants were regenerated. The plants were characterized to confirm the presence of PFP-β transgene. Plants containing the PFP-β transgene were hardened off and grown under glass house conditions. Subsequently the expression of the transgene was investigated using northern blot analysis. The results shown in FIG. 6 indicated that the transgene is expressed in all the tissues analyzed. PFP protein expression was then investigated by means of protein blot analysis. The results shown in FIG. 7 indicated that endogenous PFP levels were decreased in leafroll and internodal tissue.

Lastly, the influence of the abovementioned changes in PFP-β protein levels/activity in sucrose accumulation in the plants was determined. The results presented in Table 1 below show a substantial increase in sucrose levels in the clones with reduced PFP-β protein.

### EXAMPLES

Cloning and Characterization of a Sugarcane PFP-β cDNA Fragment

To ensure optimal antisense and/or co-suppression mediated gene silencing in sugarcane the endogenous PFP-β gene sequence was isolated. A set of degenerate primers was designed, based on the consensus sequence of the castor bean and potato PFP-β gene sequences available in the international database; GenBank accession numbers Z32850 and M55191 respectively (http://www.ncbi.nlm.nih.gov/). These primers were used to amplify a 248bp fragment from sugarcane leafroll RNA using the Titan™RT-PCR system according to the manufacturers specifications (Boehringer Mannheim). This amplified PFP-β cDNA fragment was used as a probe to screen a sugarcane leafroll cDNA library (Lambda ZAP II system, Stratagene) for putative PFP-β clones. The cDNA inserts of the isolated clones were sequenced to verify their integrity and identity. Automated DNA sequencing was performed with an Applied Biosystem ABI Prism 373 Genetic Analyzer using an ABI BigDye™ terminator cycle sequencing ready reaction kit according to the manufacturer's recommendations (Perkin-Elmer, part number 430 3152). The sequence of the insert of one such clone, PFP#5, is shown as an example in FIG. 1. Comparing this sequence to the international database confirmed its identity as PFP-β (BLASTX software; http://www.ncbi.nlm.nih.gov/cgi-bin/BLAST/). It contains an 1170 bp cDNA fragment which represents more than 50% of the PFP-β coding sequence and includes a 260 bp 3′-untranslatable sequence. The complete sugarcane PFP-β coding sequence, as presented in FIG. 2, was obtained by sequencing other cDNA- and gDNA library clones.

Construction of Plant Expression Vectors

As an example, the construction of a set of plant expression vectors for the down regulation of PFP activity in sugarcane will be described in detail. These constructs contain the PFP-β fragment from the leafroll cDNA library clone PFP#5. The 1170 bp cDNA insert was excised using the restriction enzyme EcoR I and cloned into the EcoR I site of the plant expression vector pUBI 510 (ECACC deposit reference number: 00042603) which confers high-level, constitutive gene expression in sugarcane cells. The expression vector contains two promoters, the CaMV 35S and the maize polyubiquitin UBI promoter. One of the vectors, pUSPc 510, contains the fragment in the sense orientation but it lacks a translation initiation codon and the other vector, pASPc 510, contains the fragment in the antisense orientation. Schematic representations of the two expression vectors are shown in FIGS. 3 and 4 respectively.

Transformation of Sugarcane

Sugarcane tissue culture and transformation was done as described by Snyman et al. (1996). One of the expression vectors and a selectable marker gene, neomycin phosphotransferase II (npt II), driven by the synthetic Emu promoter (Chamberlain et al., 1994), was co-transformed into sugarcane callus using a particle inflow gun (Finer et al., 1992). Transformed calli were selected on geneticin-containing medium and putative transgenic plants regenerated.

Characterization of Transgenic Sugarcane Plants

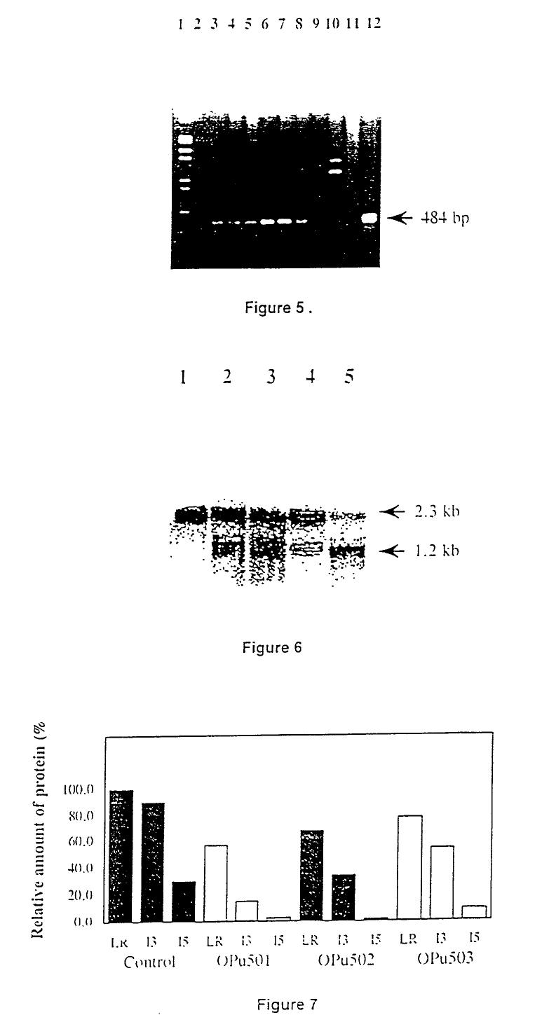

As an example, the characterization of a set of transgenic sugarcane plants transformed with the pUSPc 510 expression vector will be described in detail. These clones were designated OPu followed by a specific three digit clone number, e.g. OPu 501. As a first characterization step genomic DNA (gDNA) was extracted from putative transgenic plants and subjected to PCR analysis to confirm the presence of the PFP-β transgene before the plants were hardened off and allowed to grow under glass house conditions. FIG. 5 represents an example of a PCR analysis used to detect transgenes in regenerated sugarcane plants. PCR analysis to identify transgenic sugarcane plants. gDNA from nine putative pUSPc 510 transgenic plants (lanes 2-10) was used as template for specific PCR. The 484 bp fragment in lanes 3-8 confirms the presence of the PFP-β cDNA transgene in these clones. gDNA from an untransformed NCo 310 sugarcane plant was used as negative control (lane 11) and a positive control using the transformation vector, pUSPc 510, was also included (lane 12). In this example the amplification of a 484 bp fragment confirms the presence of the cDNA derived transgene. Due to the presence of introns the primers used will amplify a ˜1250 bp fragment from the endogenous genomic sequence (as seen in lane 10). Moreover, the genomic sequence will amplify at low efficiency because the up-stream primer, based on the cDNA sequence, overlaps an exon/intron splice site.

Subsequently the expression of the transgene on transcriptional level and its possible effect on endogenous PFP-β transcript levels was investigated using northern blot analyses. Total RNA was isolated from leafroll and young internodal tissue as described by Bugos et al. (1995). After gel electrophoresis of 20 μg total RNA per sample the RNA was transferred to Nylon™ membranes and probed with the 1170 bp PFP-β cDNA fragment. The results, as presented in FIG. 6, clearly indicate that the transgene is expressed in all the tissues analyzed. Northern blot analysis of transgenic sugarcane plants transformed with an untranslatable form of the PFP-β gene. Lane 1: Untransformed sugarcane leafroll RNA. Lane 2: Transgenic clone OPu 503 leafroll RNA. Furthermore, the result shows that some of the transgenic tissues had reduced levels of the endogenous PFP-β transcript. The untranslatable transgene transcript is represented by the 1.2 kb band and the endogenous PFP-β transcript by the 2.3 kb band. Levels of the PFP protein in the transgenic clones were compared to an untransformed control plant by means of protein blot analyses. Crude protein extracts were prepared from leafroll and young internodal tissue as described by Whittaker and Botha (1999). Ten microgram total protein of each sample was analyzed using SDS-PAGE, where after the proteins were transferred to nitrocellulose membranes and probed with potato PFP-β antiserum. A 63 kDa polypeptide cross-reacted with the antibody and the bands obtained were quantified using spot densitometry (Alphalmager 2000™ Gel Documentation and Analysis System, Alpha Innotech). The results, presented in FIG. 7, indicated that the relative level of PFP-β protein decreased in the transgenic plants. This is especially evident in the tissues with lower levels of PFP, e.g. internode 5.

Finally the influence, of the above mentioned changes in PFP-β protein levels/-activity on sugar accumulation, was determined. Sugars were extracted from internodes 7 to 13 of an untransformed control and the three OPu clones and quantified enzymatically. The results presented in Table 1 clearly shows an increase in sucrose levels in the clones with reduced PFP-β protein.

Table 1. Sugar Content of Intemodal Tissue of Transgenic Sugarcane Clones with Reduced PFP Activity.

Sugar content (umol g⁻¹ fresh weight)

| | Untransformed | | | | | | | |
|---|---|---|---|---|---|---|---|---|
| Internode | control | | OPu 501 | | OPu 502 | | OPu 503 | |
| No | Hexoses | Sucrose | Hexoses | Sucrose | Hexoses | Sucrose | Hexoses | Sucrose |
| 7 | 3.30 | 385.93 | 114.24 | 238.23 | 151.92 | 197.22 | 50.21 | 376.15 |
| 9 | 2.55 | 320.65 | 13.73 | 886.64 | 154.60 | 228.93 | 8.70 | 796.22 |
| 11 | 2.40 | 344.25 | 5.58 | 750.94 | 63.64 | 412.27 | 6.14 | 894.22 |
| 13 | 4.86 | 295.69 | 5.07 | 862.60 | 20.69 | 372.98 | 5.58 | 881.95 |

In conclusion, it was found that the isolated PFP-β gene fragments could be used to down regulate the level of active PFP in the cells and thereby manipulating sucrose metabolism in the cells.

### REFERENCES

Ausubel F A, Brent R, Kingston R E, Moore D D, Seidman J G, Smith J A, Struhl K (eds) (1998) Current Protocols in Molecular Biology. John Wiley and sons, New York.

Black C C, Loboda T, Chen J-Q, Sung S-J S (1995) Can sucrose cleavage enzymes serve as markers for sink strength and is sucrose a signal molecule during plant sink development? In: Pontis H G, Salemo G L, Echeverria E J (eds) Sucrose Metabolism: Biochemistry, Physiology and Molecular Biology. Waverley Press, Baltimore, Md., pp 49-64. ISBN 0-943088-32-3.

Botha F C, Small J G C, de Vries C. (1986) Isolation and Characterization of pyrophosphate: D-fructose-6-phosphate 1-phosphotransferase from cucumber seeds. Plant Cell Physiology 27: 1285-1295.

Botha F C, O'Kennedy M M, du Plessis S (1992) Activity of key enzymes involved in carbohydrate metabolism in Phaseolus vulgaris cell suspension cultures. Plant Cell Physiol 33: 477-483.

Bugos R C, Chiang V L, Zhang X H, Campbell E R, Podila G K, Campbell W H (1995) RNA isolation from plant tissues recalcitrant to extraction in guanidine. Biotechniques 19:734-737.

Carlisle S M, Blakeley S D, Hemmingsen S M, Trevanion S J, Hiyoshi T, Kruger N J, Dennis D T (1990) Pyrophosphate-dependent phosphofructokinase. Conservation of protein sequence between the α-and β-subunits and the ATP dependent phosphofructokinase. J Biol Chem 265: 18366-18371.

Carnal N W, Black C C (1979) Pyrophosphate-dependent 6-phosphofructokinase, a new glycolytic enzyme in pineapple leaves. Biochem Biophys Res Commun 86: 20-26.

Chamberlain D A, Brettel R I S, Last D I, Witrzwns B, McElroy D, Dolferus R, Dennis E S (1994) The use of the Emu promoter with antibiotic and herbicide resistance genes for the selection of transgenic wheat callus and rice plants. Austral J Plant Physiol 21: 95-112.

Edwards J, ap Rees T (1986) Sucrose partitioning in developing embryos of round and wrinkled varieties of Pisum sativum. Phytochemistry 25: 2027-2032.

Finer J J, Vain P, Jones M W, McMullen M D (1992) Development of the particle inflow gun for DNA delivery to plant cells. Plant Cell Rep 11: 323 -328.

Iyer L M, Kumpatla S P, Chandrasekharan M B, Hall T C (2000) Transgene silencing in monocots. Plant Mol Biol 43:323-346.

Kruger N J, Dennis D T (1987) Molecular properties of pyrophosphate: fructose-6-phosphate phosphotransferase from potato tuber. Arch Biochem Biophys 256: 273-279.

Plaxton W C (1996) The organization and regulation of plant glycolysis. Ann Rev Plant Physiol Plant Mol Biol 47: 185-214.

Snyman S J, Meyer G M, Carson D L, Botha F C (1996) Establishment of embryogenic callus and transient gene expression in selected sugarcane varieties. S Afr J Bot 62: 151-154.

Vaucheret H, Bćlin C, Elmayan T, Feuerbach F, Godon C, Morel J-B, Mourrain P, Palauqui J-C, Vemhettes S (1998) Transgene-induced gene silencing in plants. Plant J 16: 651-659.

Whittaker A, Botha F C (1999) Pyrophosphate:D-fructose-6-phosphate 1-phosphotransferase activity patterns in relation to sucrose storage across sugarcane varieties. Physiol Plant 107: 379-386.

Yan T F C, Tao M (1984) Multiple forms of pyrophosphate: D-fructose-6-phosphate 1-phosphotransferase from wheat seedlings. Regulation by fructose-2,6-bisphosphate. J Biol Chem 259: 5087-5092.

Hajirezaei M, Sonnewald U, Viola R, Carlisle S, Dennis D, Stitt M (1994) Transgenic potato plants with strongly decreased expression of pyrophosphate: fructose-6-phosphate phosphotransferase show no visible phenotype and only minor changes in metabolic fluxes in their tubers. Planta 192: 16-30.

Paul M, Sonnewald U, Hajirezaei M, Dennis D, Stitt M (1995) Transgenic tobacco plants with strongly decreased expression of pyrophosphate: fructose-6-phosphate phosphotransferase do not differ significantly from wild type in photosynthate partitioning, plant growth or their ability to cope with limiting phosphate, limiting nitrogen and suboptimal temperatures. Planta 196: 277-283.

The contents of these documents are incorporated herein by reference.

We claim:

1. A method of regulating and manipulating sucrose content in a sugar storing plant by regulating the activity of the PFP enzyme in the plant.

2. A method according to claim 1, wherein the sucrose content of the plant is increased by the down regulation of the PFP enzyme in the plant.

3. A method according to claim 2 wherein the PFP enzyme is down regulated by the introduction of an untranslatable form or an antisense form of the nucleotide sequence as set out in FIG. 1, a nucleotide sequence which is complementary to the nucleotide sequence of FIG. 1, a variant of the nucleotide sequence of FIG. 1, a portion of the nucleotide sequence of FIG. 1, or a nucleotide sequence which hybridizes to the nucleotide sequence of FIG. 1 under stringent hybridization conditions.

4. A method according to claim 3 wherein the untranslatable or antisense nucleotide sequence is introduced into the plant using a plant expression vector.

5. A method according to claim 4 wherein the plant expression vector is pUSPc 510 or pASPc 510.

6. An isolated nucleotide sequence comprising:
(i) a nucleotide sequence as set out in FIG. 1;
(ii) a nucleotide sequence which is complementary to the nucleotide sequence of (i);
(iii) a variant of the nucleotide sequence of (i);
(iv) a portion of the nucleotide sequence of (i); or (v) a nucleotide sequence which hybridizes to the nucleotide sequence of (i) under stringent hybridization conditions.

7. An isolated nucleotide sequence as set out in FIG. 2.

8. A nucleotide sequence according to claim 6 which is in an antisense orientation.

9. A gene construct comprising a promoter and nucleotide sequence as defined in claim 6 in a sense orientation, the gene construct lacking a translation initiation codon upstream of the nucleotide sequence or possessing an in-frame termination codon directly downstream of the initiation codon.

10. A gene construct according to claim 9 which comprises two promoters.

11. A gene construct according to claim 10 wherein the promoters are the CaMV35S and the maize polyubiquitin (UBI) promoters.

12. A gene construct comprising a promoter and a nucleotide sequence as defined in claim 6 in an antisense orientation.

13. A gene construct according to claim 12 which comprises two promoters.

14. A gene construct according to claim 13 wherein the promoters are the CaMV35S and the maize polyubiquitin (UBI) promoters.

15. The plant expression vector pUSPc 510 which includes the nucleotide sequence of FIG. 1 in a sense orientation, but in an untranslatable form.

16. The plant expression vector pASPc 510 which includes the nucleotide sequence of FIG. 1 in an antisense orientation.

17. A transformed plant cell which includes a gene construct according to claim 9.

18. A transgenic plant or plant part containing or derived from the transformed plant cell of claim 17.

19. A transgenic plant part according to claim 18 which is a callus.

20. A transformed plant cell according to claim 17 which is characterized by a lower level of the PFP β protein.

21. A transformed plant or plant part according to claim 18 characterized by a lower level of the PFP β protein.

22. A transformed plant cell according to claim 17 characterized by a lower level of PFP activity.

23. A transgenic plant or plant part according to claim 18 characterized by a lower level of PFP activity.

24. A transformed plant cell according to claim 17 characterized by a higher level of sucrose.

25. A transgenic plant or plant part according to claim 18 characterized by a higher level of sucrose.

26. A method of regulating or manipulating the level of active PFP in a plant cell including the step of transforming the plant cell with at least one gene construct according to claim 9.

27. A method of maintaining or increasing the sucrose level in plant tissue including the step of transforming cells of the plant tissue with at least one gene construct according to claim 9.

28. A method of manipulating sucrose metabolism in a plant cell of a sugar-storing plant including the step of co-transforming the cell with a gene construct according to claim 9.

29. A method according to claim 28 wherein the sucrose metabolism in a sugar-storing plant or sugar-storing plant part containing stored sugar is altered.

30. A method according to claim 26 wherein the plant is sugarcane.


SEQUENCE LISTING (2 sequences)

SEQ ID NO: 1 - DNA, 1176 bp, Saccarum officinarum
gaattccgatttagcctcatactgcttctcacattacattgggatgcgctttgcaaacacaccccaatgctgcactcattggggaagaggttgctgcgaagaagcaaacccttaagaacgtcacaaactacattactgatatcatctgcaagcgtgcagatcttggttacaactatggggttatccttataccagaaggcctgattgatttcatcccagaggttcaaaaactcatcgcagaattgaatgaaattttggcacatgatgtggttgatgaggcaggggcctggaaaagcaagcttcagcctgaatcaaaggagctgtttgagtttttgcccaaaactattcaggagcaacttatgcttgaaaggggcccccatggcaatgttcaggttgcaaaaattgaaaccgagaaaatgcttattagcatggtggaaactgaactggagaagagaaaagcagaggggagatactctgcacatttcagagggcaagctcatttctttgggtacgaaggaagatgtggccttcctaccaattttgattctaactattgctatgcattaggctatggtgctggtgcccttctccaaagtgggaagacaggacttatttcatcggttggcaaccttgcggctccagtagaagaatggactgttggtggaacagcattgacatcactgatggatgttgagaggaggcatggcaagttcaagccagtgatcaagaaggctatggtggaacttgatgctgcacctttcaagaaatatgcatcaatgcgggatgagtgggccaccaagaacagatacatcagccctggccccatccagttcagtggccctggaagtgatgactcgaaccacactttgatgctggaactcggtgctgagttatagagatgcgtcctttgcttatttttgtttcttacagttttgggagtggagactggacactgggtctcctggagcagcctgcagtctccatattgtgaattgtttaataagaggttcgatgtgagttttctgcgtagcggactggatgtagcaaataagaactggttttagcattttttgtatgatttacgcaccaactgacttgtcttgtaaccctgattctgttccactggttgcaatctcgtgagaatgaacaagttgatatgaggctaaatcggaattc

SEQ ID NO: 2 - DNA, 1668 bp, Saccharum officinarum
atggcggcgccgagcggaccatcacctgggactgggaggttggcgtcggtttacagcgaggtgcagacgagccgcctccatcacgcgatccggctcccctccgtcctctgctcccaattctccctcgtcgatggacctcccagctcagccacggggaacccggatgagatcgcgaagctgttccctaacttgtttgggcagccgtcggcgacattggtgccggccaaagaggcggtggaggggaaggcgctgaaggtcggggtggtgctctctggtggacaagcacccggtgggcacaatgtgatctgcggtatcttcgatttcttgcagaaacacgcaaagggaagcacaatgtatggattcaaaggaggcccagcaggggtgatgaagtgcaagtacgtcaaactcaataccgatttcgtctatccctacagaaaccagggtggttttgatatgatctgtagtggaagggataagattgaaacaccagagcagtttaagcaagccgaagatacagccaacaaacttgagttggacggacttgttgttattggacgggacgattcaaatactcatgcttgcctctttgctgaatacttcaggagtaaaaatttgaaaacccgtgtcattggctgcccaaagaccattgatggtgatctcaaatgcaaagaggttccaaccagttttggatttgacactgcatgcaagatctattcagaaatgattggaaatgtcatgattgatgcccgatcaactggaaaatattatcactttgtacggcttatggggcgtgctgcttctcacattacattgggatgcgctttgcaaacacaccccaatgctgcactcattggggaagaggttgctgcaaagaagcaaacccttaagaacgtcacaaactacattactgatatcatctgcgagcgtgcagatcttggttacaactatggtgttatccttataccagaaggcctgattgatttcatcccagaggtgcagaatatcattgctgaattgaatgaaattttggcacatgatgttgttgatgaggcaggggcctggaaaagcaagcttcagcctgaatcaaaggagctgtttgagtttttgcccaaaactattcaggagcaacttatgcttgaaaggggcccccatggcaatgttcaggttgcaaaaattgaaaccgagaaaatgcttattagcatggtggaaactgaactggagaagagaaaagcagaggggagatactctgcacatttcagagggcaagctcatttctttgggtacgaaggaagatgtggccttcctaccaattttgattctaactattgctatgcattaggctatggggctggtgcccttctccaaagtgggaagacaggacttatttcatcggttggcaaccttgcggctccagtagaagaatggactgttggtggaacagcattgacatcactgatggatgtggagaggaggcatggcaagttcaagccagtgatcgagaaggctatggtggaacttgatgctgcacctttcaagaaatatgcatcaatgcgggatgagtgggccaccaagaacagatacatcagccctggccccatccagttcagtggccctggaagtgatgactcgaaccacactttgatgctggaactcggtgctgagttatag